(19) United States
(12) Patent Application Publication
(10) Pub. No.: US 2005/0043247 A1
(43) Pub. Date: Feb. 24, 2005

(54) Spray-dried amorphous BIBN 4096, process for preparing and the use thereof as inhalative

(75) Inventors: Michael Trunk, Ingelheim (DE); Claudius Weiler, Ingelheim-Grosswinternheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/902,305

(22) Filed: Jul. 29, 2004

Related U.S. Application Data
(60) Provisional application No. 60/503,014, filed on Sep. 15, 2003.

(30) Foreign Application Priority Data
Aug. 18, 2003 (DE) 103 38 402.2

(51) Int. Cl. A61K 38/04; A61K 31/517; A61L 9/04; A61K 9/14

(52) U.S. Cl. 514/19; 514/252.17; 424/46

(57) ABSTRACT

The invention relates to the CGRP antagonist 1-[N²-[3,5-dibromo-N-[[4-(3,4-dihydro-2 (1H)-oxoquinazolin-3-yl)-1-piperidinyl]carbonyl]-D-tyrosyl]-L-lysyl]-4-(4-pyridinyl)-piperazine (A) and the physiologically acceptable salts thereof which are stable in the amorphous state under normal conditions (T<50° C., relative humidity<75%) and are in the form of microparticles, processes for preparing such microparticles from these substances and the use of these particles for preparing a pharmaceutical composition of the inhalable powder type for pulmonary and nasal inhalation, particularly for preparing a pharmaceutical composition for the treatment of headaches, migraine and cluster headache.

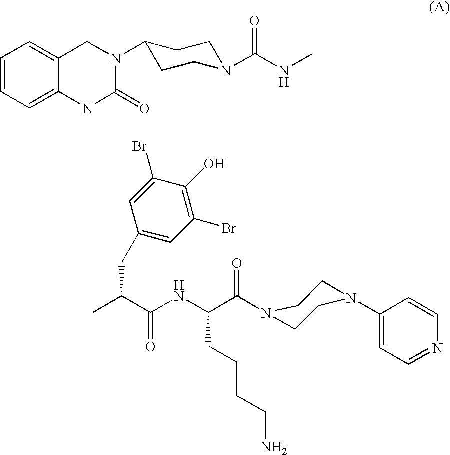

The invention relates to the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof which are stable in the amorphous state under normal conditions (T<50° C., relative humidity<75%) and are in the form of microparticles, processes for preparing such microparticles from these substances and the use of these particles for preparing a pharmaceutical composition of the inhalable powder type for pulmonary and nasal inhalation, particularly for preparing a pharmaceutical composition for the treatment of headaches, migraine and cluster headache.

### BACKGROUND TO THE INVENTION

The CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) is known from International Patent Application PCT/EP97/04862 (published as WO 98/11128) and has the following structure:

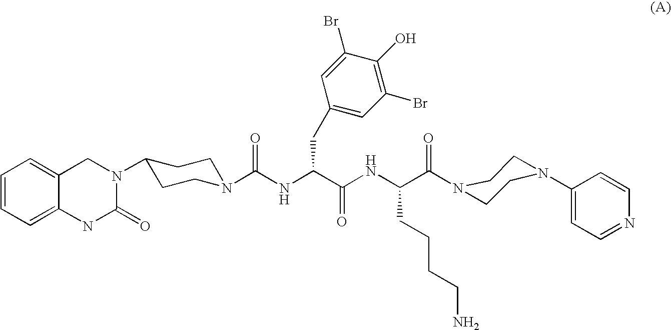

### PRIOR ART

The active substance base (A) is a highly effective CGRP antagonist for the acute and prophylactic treatment of headaches, particularly migraine and cluster headache, which cannot be administered orally using conventional formulations, as the substance has very limited oral bioavailability.

For treating attacks of migraine it is essential that an active substance is systemically available as quickly as possible. The treatment should be uncomplicated for the patient to administer and no other conditions which could affect bioavailability (e.g. the food effect) should restrict the use of the medicament for the patient.

Active substances which are intended to be systemically available are usually administered by oral route. If this route is unsuitable or undesirable on account of particular properties of the active substance or particular demands made of the application, other possible ways of administering substances systemically are known in the art. For example, inhalation, by means of which active substances may be administered systemically as well as topically, has been under discussion for some time. For substances which prove critical on account of their decomposition in solution or which have poor solubility per se, powder inhalation is an option. The absolute amount of the active substance which has to be administered per application makes particular demands of the formulation. On the other hand, the physical stability (e.g. aerodynamic particle size, dispersibility, physicochemical properties) of the active substance has proved to be a critical requirement for the development and production of an inhalable powder.

With formulations of the powder inhalant type, inhalable powders, which are packaged for example in suitable capsules (inhalettes), are delivered to the lungs by means of powder inhalers. Similarly, other systems in which the quantity of powder to be administered is pre-dosed (e.g. blisters),are also known as multidose powder systems. Alternatively, the medicament may also be inhaled by the use of suitable powdered inhalable aerosols which are suspended for example in HFA134a, HFA227 or mixtures thereof as propellant gas.

In powder inhalation, the microparticles of a pure active substance are administered through the airways onto the surface of the lungs, e.g. in the alveoli, by the inhalation process. These particles settle on the surface and can only be absorbed into the body after the dissolving process by active and passive transporting processes.

Inhalation systems are known in the literature in which the active substance is present in the form of solid particles either as a micronised suspension in a suitable solvent system as carrier or in the form of a dry powder.

Usually, powder inhalants, e.g. in the form of capsules for inhalation, are prepared on the basis of the general teaching as described in DE-A-179 22 07.

A critical factor in multi-substance systems of this kind is the uniform distribution of the pharmaceutical composition in the powder mixture.

The pharmaceutical active substance used to prepare the above-mentioned pharmaceutical composition should be as pure as possible and its stability on long-term storage must be guaranteed under different environmental conditions. This is absolutely essential in order to prevent the use of pharmaceutical compositions in which breakdown products, for example, are present together with the active substance itself.

Apart from the requirements concerning chemical stability of the active substance s outlined above it must be generally borne in mind that any change to the solid state of a pharmaceutical composition or to the active substance used which improves its physical and chemical stability gives a considerable advantage over less stable forms of the same pharmaceutical composition. Different physical/physicochemical properties may, however, bring about improved pharmacological/pharmacokinetic properties of the pharmaceutical composition in some cases. In particular, depending on the formulation, special morphological properties of solid particles may be beneficial to the preparation of a pharmaceutical composition.

It is known from the literature that particles in the submicron range can be produced by spray-drying. Usually, industrially suitable formulations which exhibit sufficient dispersibility in medical use (inhalation) may be prepared from spray-dried particles of this kind in accordance with the method cited above (DE-A-179 22 07) [Y.-F. Maa, P.-A. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P. A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109].

It is also known from the literature that using special methods it is possible to produce so-called “large porous particles” which have proved particularly suitable for use in powder inhalants (D. A. Edwards, J. Hanes, G. Caponetti, J. Hrkach, A. Ben-Jebria, M. L. Eskew, J. Mintzes, D. Deaver, N. Lotan, R. Langer, Science, 276 (1997) 1868-1871). By these are meant particles with a mean geometric size of more than 5 μm (e.g. 8.5 μm to 20 μm), which behave aerodynamically in the same way as particles less than 5 μm in size, these powders also being characterised by an extremely low density (<0.4 g/cm³).

### STATEMENT OF THE PROBLEM

The complex problem of the present invention was thus primarily to provide novel stable microparticles of the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof, which meet the stringent requirements mentioned above that are imposed on a pharmaceutical active substance for a powder inhalant for pulmonary and nasal inhalation and compared with conventional micronised starting material (obtained e.g. by air-jet grinding) have proved suitable for use as powder inhalants in terms of their pharmacological/pharmacokinetic properties. According to the invention the morphology of the microparticles was to be optimised so that the formulation consisting thereof preferably contains no excipient and hence consists exclusively of active substance.

The formulation according to the invention should also exhibit a rapid onset of activity for the treatment of the acute pain which occurs very suddenly in the case of migraine. This means that rapid absorption of the active substance and a rapid increase in the plasma level must be guaranteed.

### DETAILED DESCRIPTION OF THE INVENTION

A rapid onset of activity for the treatment of acute pain as well as a high plasma level of the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof within a very short time can best be achieved through the lungs as the site of absorption.

It has been found that when the active substance (A) is administered by inhalation in the form of a powder inhalant a bioavailability of about 60% can be achieved based on the fine content of the formulation (corresponding to FPD “fine particle dose”, determined according to USP 24 Suppl. 2000).

The present invention therefore consists in the preparation of novel, stable microparticles of the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof, which are surprisingly especially suitable for preparing powder inhalants for pulmonary and nasal inhalation.

They are characterised by special physical and physicochemical properties, which lead to an improved pharmacological as well as pharmacokinetic activity when the substance is inhaled. One surprising feature is that by varying/optimising the particle shape of these particles with a large specific surface area, the aerodynamic properties and also the increase in dispersibility and inhalability can be improved.

The invention also includes the chief method of preparing microparticles of this kind and the use thereof for preparing pharmaceutical compositions in the form of a powder inhalant.

According to the invention in addition to the active substance base the corresponding physiologically acceptable acid addition salts are used which are selected for example from among 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate, while the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, the sulphate and the hydrobromide are preferred and the 1-[N²-[3,5-dibromo-N-[[4-(3,4-dihydro-2(1H)-oxoquinazolin-3-yl )-1-piperidinyl]carbonyl]-D-tyrosyl]-L-lysyl]-4-(4-pyridinyl)-piperazine hydrochloride as well as the free active substance base are particularly preferred.

Particle geometries of the microparticles which have proved advantageous may be described as collapsed hemispheres and have a crinkled structure. In terms of geometry, particles prepared by the processes described below have particle shapes which may be described, depending on the test conditions, between the extremes of “spherical shell fragment”, “thin-walled, totally collapsed sphere”, “crinkled, filigree-flaked platelet structure”, as well as “rosette-like crinkled structure”.

These particles are characterised in that
(a) they have a specific surface area between 3 m²/g and 35 m²/g, preferably between 5 m²/g and 30 m²/g and particularly preferably between 10 m²/g and 30 m²/g,
(b) the characteristic Q_{(5.8)} is between 50% and 100% and
(c) the parameter X₅₀ is in the range from 0.5 μm to 10 μm, preferably from 0.5 μm to 6 μm.

The crinkled microparticles according to the invention are suitable for preparing powder inhalants for pulmonary and nasal inhalation, in which no other excipients or additives (carrier materials) are needed in order to obtain an industrially workable powder which can be further processed directly and which has excellent properties in terms of dispersibility and is sufficiently easy to process with regard to its cohesive properties.

In a first aspect the present invention thus relates to a powder inhalant for pulmonary and nasal inhalation, comprising the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or one of the physiologically acceptable salts thereof in the form of crinkled microparticles, characterised in that
(a) they have a specific surface area between 3 m²/g and 35 m²/g, preferably between 5 m²/g and 30 m²/g and particularly preferably between 10 m²/g and 30 m²/g,
(b) the characteristic Q_{(5.8)} is between 50% and 100% and
(c) the parameter X₅₀ is in the range from 0.5 μm to 10 μm, preferably from 0.5 μm to 6 μm.

The crinkled microparticles according to the invention are however also suitable for preparing powder inhalants wherein the active substance is administered together with an excipient.

Normal carrier materials or flow adjuvants may be used as physiologically acceptable homogeneous excipients according to the invention. The normal carrier materials may be selected from among the monosaccharides (e.g. glucose or arabinose), disaccharides (e.g. lactose, saccharose, maltose, trehalose), oligo- and polysaccharides (e.g. dextrans, starch, cellulose derivatives), polyalcohols (e.g. mannitol, sorbitol, xylitol), salts (e.g. sodium chloride, calcium carbonate), polylactides, polyglycolides and mixtures of these excipients. The flow adjuvants may for example be selected from a group consisting of magnesium stearate, calcium stearate, stearic acid, stearyl alcohols, calcium behenate, calcium arachinate, hydrogenated vegetable oils such as for example hydrogenated castor oil or hydrogenated cottonseed oil, fatty acid esters, sodium stearyl fumarate, sodium dodecyl sulphate, magnesium dodecyl sulphate and mixtures of these flow adjuvants.

The method of preparing the microparticles according to the invention is characterised in that the active substance is suitably dissolved, sprayed and dried in a spraying tower. The particle morphology including the particle size of these microparticles can be deliberately controlled by the choice of process parameters and production parameters.

In a second aspect the present invention thus relates to a process for producing the microparticles of the active substance base (A) according to the invention, comprising the following steps:
(a) dissolving the active substance (A) in an organic solvent or an organic-aqueous solvent mixture to prepare a sprayable solution with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
   (i) an entry temperature of the drying gas from 100° C. to 350° C., preferably from 120° C. to 250° C. and particularly preferably from 130° C. to 200° C. and
   (ii) an exit temperature of the drying gas from 40° C. to 120° C. and
(d) separating the dried solid fraction from the current of drying gas in the usual way.

Preferably the microparticles of the active substance base (A) according to the invention are prepared by a method comprising the following steps:
(a) dissolving the active substance (A) in an organic solvent or an organic aqueous solvent mixture in order to prepare a sprayable solution with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way with a flow volume of spray gas of from 1 Nm³/h to 15 Nm³/h, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
   (i) an entry temperature of the drying gas from 100° C. to 350° C., preferably from 120° C. to 250° C. and particularly preferably from 130° C. to 200° C.,
   (ii) an exit temperature of the drying gas from 40° C. to 120° C. and
   (iii) a flow volume of the drying gas from 15 Nm³/h to 150 Nm³/h and
(d) separating the dried solid fraction from the current of drying gas in the usual way.

Organic solvents, organic-aqueous solvent mixtures and water have proved suitable as solvents for preparing a sprayable solution of the active substance base. Preferably an alcoholic or alcoholic-aqueous solvent system is used, particularly preferably a solvent mixture consisting of ethanol/methanol/water or ethanol/propanol/water and most particularly preferably the solvent mixture of ethanol/water or the solvent absolute ethanol, methanol or water.

In a third aspect the present invention relates to a process for preparing the microparticles of the salts of the active substance base (A) according to the invention, comprising the following steps:
(a) dissolving the active substance base (A) in water or an aqueous buffer system and adding the corresponding acid in order to prepare a sprayable salt solution of the active substance with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
   (i) an entry temperature of the drying gas from 100° C. to 350° C., preferably from 120° C. to 250° C. and particularly preferably from 130° C. to 200° C. and
   (ii) an exit temperature of the drying gas from 40° C. to 120° C. and
(d) separating the dried solid fraction from the current of drying gas in the usual way.

Preferably the microparticles of the salts of the active substance base (A) according to the invention are prepared by a method comprising the following steps:
(a) dissolving the active substance base (A) in water or an aqueous buffer system and adding the corresponding acid in order to prepare a sprayable salt solution of the active substance with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way with a flow volume of spray gas of from 1 Nm³/h to 15 Nm³/h, so as to obtain a spray mist with a droplet size having the characteristic X₅₀ from 1 to 50 μm, preferably from 1 μm to 30 μm, particularly preferably from 1 μm to 20 μm, while applying the following parameters:
(c) drying the spray mist thus obtained using a drying gas while applying the following parameters:
   (i) an entry temperature of the drying gas from 100° C. to 350° C., preferably from 120° C. to 250° C. and particularly preferably from 130° C. to 200° C.,
   (ii) an exit temperature of the drying gas from 40° C. to 120° C. and
   (iii) a flow volume of the drying gas from 15 Nm³/h to 150 Nm³/h and
(d) separating the dried solid fraction from the current of drying gas in the usual way.

Water or an aqueous buffer system with a pH between 6 and 8 have proved suitable as solvents for preparing a sprayable solution of the salt forms of the active substance base (A). The active substance according to the invention which is present in the form of the free base is dissolved in an aqueous solution, which is combined with 0.9 to 1.1 equivalents of acid, according to the quantity of active substance to be dissolved, in the form of the corresponding salt. The acids according to the invention are preferably inorganic acids (for example hydrochloric acid, hydrobromic acid, phosphoric acid, nitric acid, sulphuric acid, carbonic acid), fruit acids (for example citric acid, malic acid, tartaric acid, lactic acid, succinic acid, gluconic acid), carboxylic acids (for example formic acid, acetic acid, propionic acid, hexanoic acid) as well as other organic acids such as oxalic acid, methanesulphonic acid, p-toluenesulphonic acid, fumaric acid, mandelic acid or maleic acid; it is particularly preferable to use hydrochloric acid, hydrobromic acid or sulphuric acid and particularly hydrochloric acid.

The surface qualities of the particles can be optimised by adjusting the ratio between the droplet size and solids concentration. Normally a concentration of between 0.2 and 4 wt. %, preferably between 0.2 and 3 wt. %, most preferably between 0.3 and 2 wt. % is selected.

The droplet size is a crucial parameter in the production of inhalable particles. Depending on the nozzle used the throughput of spray gas combined with the throughput of solution should be selected to achieve the desired droplet size. As there are a number of combinations of the parameters nozzle/throughput of spray gas/throughput of solution leading to a suitable droplet size, the process can usefully be defined by the droplet size which is obtained with the same nozzle parameters with water at ambient temperature. These may be described by the characteristic X₅₀ (median value of droplet size below which 50% by volume of the droplet fraction falls), which should be in the range from 1 μm to 50 μm.

The critical characteristics which impinge on the drying step are the entry and exit temperature of the drying gas, as well as the flow volume of the drying gas passing through.

In a fourth aspect the present invention relates to the use of the crinkled microparticles produced by the processes described hereinbefore for the production of a powder inhalant.

In a fifth aspect the present invention relates to the microparticles according to the invention, which can be obtained according to the processes described above.

Experimental Section

### 1) Methods of Measurement

a) Determining the Particle Size by Laser Diffraction (Frauenhofer Diffraction):

Measuring method: In order to determine the particle size the powder is fed into a laser diffraction spectrometer using a dispersing unit. The median value X₅₀ refers to the particle size below which 50% of the quantity of particles fall. The Q_{(5.8)} value describes the percentage of particles which are less than 5.8 μm in size.

Measuring device: Laser diffraction spectrometer (HELOS), Messrs. Sympatec

Software: WINDOX 4

Dispersing unit: RODOS/dispersing pressure: 3 bar

Focal length: 100 mm [measuring range: 0.9 . . . 175 μm]

Evaluation method: HRLD (V 4)

b) Determining the Specific Surface Area:

Measuring method: The specific surface is determined by exposing the powder sample to a nitrogen atmosphere at different pressures. Cooling the sample causes the nitrogen molecules to be condensed on the surface of the particles. The quantity of condensed nitrogen is determined by means of the drop in pressure in the system and the surface of the sample is calculated by means of the surface nitrogen requirement and the weight of the sample.

Measuring device: Tri Star Multi Point BET, Messrs. Micromeritics

Heating station: VacPrep 061, Messrs. Micromeritics

Heating: approx. 12 h /40° C.

Analysis parameters

| | |
|---|---|
| sample tube: | ½ inch; with filler rod |
| analysis method: | 16 point BET surface measurement |
| | 0.05 to 0.20 p/p0 |
| absolute pressure tolerance: | 5.0 mm Hg |
| relative pressure tolerance: | 5.0% |
| evacuation rate: | 50.0 mm Hg/second |
| evacuation threshold: | 10.0 mm Hg |
| evacuation time: | 0.1 h |
| free space: | lower Dewar, t: 0.5 h |
| retention time: | 20 seconds |
| minimum equilibration delay: | 600 seconds |
| adsorptive: | nitrogen |

c) Determining the Droplet Size by Laser Diffraction (According to Mie):

Measuring device: Laser diffraction spectrometer (HELOS), Messrs. Sympatec

Software: WINDOX 4

Focal length: 100 mm [measuring range: 0.9 . . . 175 μm]

Measuring method: The droplet size is determined by removing the nozzle from the spray dryer and placing the spray in the upper third of the spray cone in the centre of the laser beam. Measuring is done at ambient temperature with water as reference medium under otherwise identical conditions.

### 2) Examples of Spray Parameters

### EXAMPLE

### Spray Parameters Suitable for an Alcoholic Solution of (A) (NIRO Spray Dryer SD Micro)

| | |
|---|---|
| Concentration solution | 0.5 g (A) in 100 mL ethanol |
| Droplet size X₅₀ | 11 μm |
| (Reference solution: H₂O at ambient | |
| temperature) | |
| Flow volume “spray rate” | 4.5 mL/min |
| Spray pressure | 0.7 bar overpressure (N₂) |
| (nozzle type) | (Niro spray nozzle 0.5 mm, |
| | Art.-Nr. 248709/A) |
| Flow volume “Atomising pressure” | 2.2 kg/h |
| (nozzle type) | (Niro spray nozzle 0.5 mm, |
| | Art.-Nr. 248709/A) |
| entry temperature | 149° C. |
| exit temperature | 96° C. |
| Flow volume of “drying gas” | 20.1 kg/h |
| cross section of drying tower | 200 mm |

### 3) Characterisation of the Solid Particles Obtained in the Above Example

### EXAMPLE

| | | |
|---|---|---|
| Particle size | X₅₀ | 1.2 μm |
| | Q_{(5.8)} | 99.5% |
| Specific surface area | Sₘ | 21.5 m²/g |

### BRIEF DESCRIPTION OF THE FIGURES

FIGS. 1 and 2 show photographs of microparticles of the active substance base (A), prepared from an alcoholic spray solution by the process according to the invention.

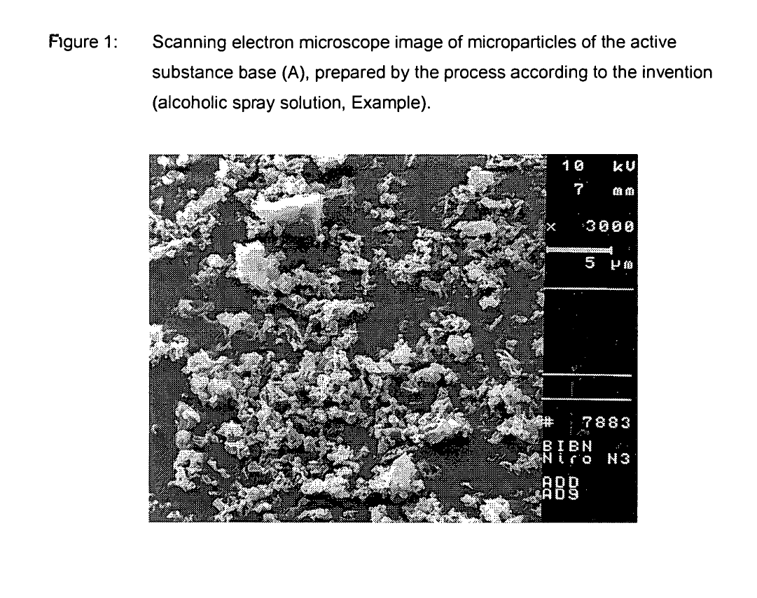

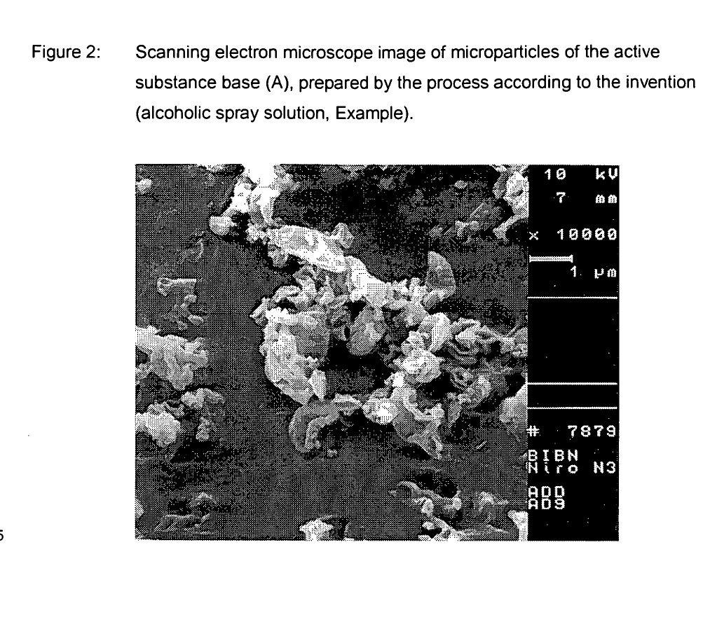

1. A powder inhalant, comprising the active substance base 1-[N²-[3,5-dibromo-N-[(4-(3,4-dihydro-2(1H)-oxoquinazolin-3-yl)-1-piperidinyl]carbonyl]-D-tyrosyl]-L-lysyl]-4-(4-pyridinyl)-piperazine 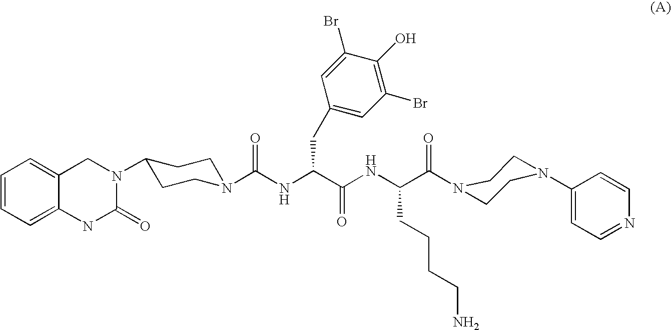
or one of the physiologically acceptable salts thereof, in the form of crinkled microparticles, characterised in that
(a) they have a specific surface area between 3 m²/g and 35 m²/g, preferably between 5 m²/g and 30 m²/g, particularly preferably between 10 m²/g and 30 m²/g,
(b) the characteristic Q_{(5.8)} is between 50% and 100% and
(c) the parameter X₅₀ is in the range from 0.5 μm to 10 μm, preferably from 0.5 μm to 10 μm.

2. The powder inhalant according to claim 1, characterised in that the active substance is the free base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A).

3. The powder inhalant according to claim 1, characterised in that the active substance is selected from the group consisting of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate.

4. The powder inhalant according to claim 1, characterised in that the active substance is selected from the group consisting of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate and hydrobromide.

5. the powder inhalant according to claim 1, characterised in that the active substance is 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride.

6. The powder inhalant according to claim 1, characterised in that it is administered together with one or more physiologically acceptable carrier materials and/or flow adjuvants.

7. The powder inhalant according to claim 6, characterised in that the carrier materials used are monosaccharides, disaccharides, oligo- and polysaccharides, polyalcohols, salts, polylactides, polyglycolides or mixtures of these carrier materials.

8. The powder inhalant according to claim 6, characterised in that the carrier materials used are glucose, arabinose, lactose or saccharose, maltose, trehalose, dextrans, starch, cellulose derivatives, mannitol, sorbitol, xylitol, sodium chloride, calcium carbonate, polylactides, polyglycolides or mixtures of these carrier materials.

9. The inhalable powder according to claim 6, characterised in that the flow adjuvants used are magnesium stearate, calcium stearate, stearic acid, stearylalcohols, calcium behenate, calcium arachinate, hydrogenated vegetable oils, fatty acid esters, sodium stearyl fumarate, sodium dodecyl sulphate, magnesium dodecyl sulphate or mixtures of these flow adjuvants.

10. A process for preparing the crinkled microparticles of the active substance base (A) according to claim 1, comprising the following steps:
(a) dissolving the active substance (A) in an organic solvent or an organic-aqueous solvent mixture to prepare a sprayable solution with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
(i) an entry temperature of the drying gas from 100° C. to 350° C., preferably from 120° C. to 250° C. and particularly preferably from 130° C. to 200° C. and
(ii) an exit temperature of the drying gas from 40° C. to 120° C. and
(d) separating the dried solid fraction from the current of drying gas in the usual way.

11. A process for preparing the crinkled microparticles of the active substance base (A) according to claim 1, comprising the following steps:
(a) dissolving the active substance (A) in an organic solvent or an organic aqueous solvent mixture in order to prepare a sprayable solution with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way with a flow volume of spray gas of from 1 Nm³/h to 15 Nm³/h, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
(i) an entry temperature of the drying gas from 100° C. to 350° C., preferably from 120° C. to 250° C. and particularly preferably from 130° C. to 200° C.,
(ii) an exit temperature of the drying gas from 40° C. to 120° C. and
(iii) a flow volume of the drying gas from 15 Nm³/h to 150 Nm³/h and
(d) separating the dried solid fraction from the current of drying gas in the usual way.

12. A process for preparing the crinkled microparticles of the salts of the active substance base (A) according to claim 1, comprising the following steps:
(a) dissolving the active substance base (A) in water or an aqueous buffer system and adding the corresponding acid in order to prepare a sprayable salt solution of the active substance with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
(i) an entry temperature of the drying gas from 100° C. to 350° C., preferably from 120° C. to 250° C. and particularly preferably from 130° C. to 200° C. and
(ii) an exit temperature of the drying gas from 40° C. to 120° C. and
(d) separating the dried solid fraction from the current of drying gas in the usual way.

13. A process for preparing the crinkled microparticles of the salts of the active substance base (A) according to claim 1, comprising the following steps:
(a) dissolving the active substance base (A) in water or an aqueous buffer system and adding the corresponding acid in order to prepare a sprayable salt solution of the active substance with a concentration of active substance of between 0.2 and 4 wt. %, preferably between 0.2 wt. % and 3 wt. %, particularly preferably between 0.3 wt. % and 2 wt. %,
(b) spraying the active substance solution thus obtained in the usual way with a flow volume of spray gas of from 1 Nm³/h to 15 Nm³/h, so as to obtain a spray mist with a droplet size having the characteristic X₅₀ from 1 to 50 μm, preferably from 1 μm to 30 μm, particularly preferably from 1 μm to 20 μm,
(c) drying the spray mist thus obtained using a drying gas while applying the following parameters:
(i) an entry temperature of the drying gas from 100° C. to 350° C., preferably from 120° C. to 250° C. and particularly preferably from 130° C. to 200° C.,
(ii) an exit temperature of the drying gas from 40° C. to 120° C. and
(iii) a flow volume of the drying gas from 15 Nm³/h to 150 Nm³/h and
(d) separating the dried solid fraction from the current of drying gas in the usual way.

14. Microparticles made by the process of claim 10, 11, 12 or 13.
